(19) United States
(12) Patent Application Publication
(10) Pub. No.: US 2024/0358993 A1
(43) Pub. Date: Oct. 31, 2024

(54) CONTROL DEVICE FOR A BLOOD PUMP

(71) Applicant: Berlin Heart GmbH, Berlin (DE)

(72) Inventor: Eike Schärff, Berlin (DE)

(73) Assignee: Berlin Heart GmbH, Berlin (DE)

(21) Appl. No.: 18/644,778

(22) Filed: Apr. 24, 2024

(30) Foreign Application Priority Data
Apr. 26, 2023 (EP) 23170017.0

(51) Int. Cl. A61M 60/122

(52) U.S. Cl. CPC A61M 60/122; A61M 2205/04; A61M 2205/10; A61M 2205/8206

(57) ABSTRACT

A control device for a blood pump is provided. The blood pump control device includes an electronic control unit and an energy storage detachably connectable to the electronic control unit via a plug connection. The plug connection is configured to establish mechanical and electrical connections between the electronic control unit and the energy storage. The plug connection includes a frame of a housing and a frame insertion element. The frame insertion element is insertable into the frame in an insertion direction in at least two orientations that differ by a relative rotation between the frame and the frame insertion element about the insertion direction. The frame and the frame insertion element have one or more electrical coupling pairs to establish the electrical connection and are arranged to establish the electrical connection between the energy storage and the electronic control unit in the two or more orientations.

### PRIORITY

This application claims priority to EP Application No. 23 170 017.0, filed on Apr. 26, 2023, the entirety of which is fully incorporated by reference herein.

### TECHNICAL FIELD

This disclosure relates to a control device for a blood pump which comprises an electronic control unit and an energy storage unit which are detachably connectable to one another electrically and mechanically via a plug connection. Furthermore, the present disclosure relates to a blood pump device which comprises the described control device.

### BACKGROUND

In the field of medical technology, increased safety and operability requirements are placed on plug connections that connect, for example, an electric battery to an electronic control unit. Various realizations of such plug connections are already known from the prior art.

EP 2 941 281 A2, for example, describes a control system for driving an implantable blood pump, comprising a first housing, with electronic components configured to drive the pump, and a second housing containing a battery. The first and second housings comprise a plurality of locking elements.

### SUMMARY

The aim of this disclosure is to provide a control device for a blood pump having an improved plug connection.

The control device comprises an electronic control unit and an energy storage unit which are detachably connectable to one another electrically and mechanically via a plug connection. Here, the plug connection for establishing a mechanical connection has a frame and a frame insertion element insertable into the frame along an insertion direction, said frame and frame insertion element being formed by a housing of the electronic control unit and a housing of the energy storage unit. Furthermore, the frame and the frame insertion element are configured such that the frame insertion element is insertable into the frame in two or more orientations which differ from one another by a relative rotation between the frame and the frame insertion element about the insertion direction. In addition, the frame and the frame insertion element have one or more electrical coupling pairs for establishing an electrical connection between the electronic control unit and the energy storage unit, and the one or more electrical coupling pairs are arranged such that an electrical connection is possible in all orientations.

The control device described has been developed based on the insight that it may be advantageous if detachably connectable units of a control device by means of a plug connection formed by a housing of the respective unit may be advantageous. This usually results in the control device having compact housing dimensions in the connected state. Connecting the two units using a cable, for example, would not result in such a compact design.

The described plug connection of the control device is typically advantageous in order to enable the energy storage to be changed safely. This is achieved in particular by providing a mechanical and electrical connection between the electronic control unit and the energy storage for all orientations that are recognizable from the outside by a user through the frame and frame insertion element. This is particularly advantageous in situations where the energy storage needs to be changed quickly.

Based on the control device described, further possible examples of the control device are described below.

In general, the frame may take various forms. In one example, the frame has an approximately rectangular basic shape. In another example, the frame has an approximately round basic shape, in a further example an approximately square basic shape.

In most examples of the control device, the energy storage unit comprises an accumulator for storing electrical energy. In some examples, the energy storage unit additionally or alternatively comprises a battery.

In addition, in most examples, the control device, in the connected state, has an overall housing which is at least partially formed by the housing of the electronic control unit and the energy storage unit.

In another example, the frame element and the frame insertion element additionally or alternatively have one or more mirror axes in a plane perpendicular to the insertion direction. Furthermore, the electrical coupling pairs are arranged symmetrically with respect to the one or more mirror axes. A symmetrical arrangement of the electrical coupling pairs is typically advantageous in order to prevent jamming when inserting and removing the frame insertion element into and from the frame. An asymmetrical arrangement of the coupling pairs is in principle also an example of the invention. However, the different force distribution along the insertion direction during removal or insertion more easily leads to the frame insertion element becoming jammed in the frame.

In a further example of the control device, each coupling pair additionally or alternatively has an electrical contact arranged on the electronic control unit and an electrical mating contact arranged on the energy storage unit. In variants of this example, the electrical contact and the electrical mating contact are formed by a pin and a socket, respectively. In another variant, the pin is additionally arranged in the frame on the electronic control unit and the socket on the frame insertion element of the energy storage unit. This variant is typically particularly safe, as there are no exposed live contacts. In a further variant, the contact is additionally or alternatively arranged on a rear wall within the frame and/or the mating contact is arranged on a front of the frame insertion element.

In a further example of the control device, the plug connection is additionally or alternatively configured to transmit a plurality of different electrical quantities between the electronic control unit and the energy storage unit, and the plug connection comprises two electrical coupling pairs for the transmission of at least one of the different electrical quantities. This is usually advantageous in order to create redundancy in the event of a failure of one of the coupling pairs and to increase patient safety. In one variant, the plug connection comprises two electrical coupling pairs for the transmission of each electrical quantities. In this context, electrical quantities include electrical signals, such as bus signals, as well as ground voltage, phase voltage, etc.

In one example of the control device, the plug connection additionally or alternatively has at least two electrical coupling pairs, wherein the coupling pairs are configured such that, when the frame insertion element is inserted into the frame, an electrical connection is established by one of the two coupling pairs before an electrical connection is established by another coupling pair. This is typically advantageous in order to avoid undefined states in an electrical circuit of the control device. In one variant, elements of the coupling pair, such as contact or mating contact, are for this purpose arranged in a leading position in a plane perpendicular to the insertion direction. In another variant, for example, one element of the coupling pair for ground is arranged in a leading position. In another variant, an element of the coupling pair for the supply voltage is additionally or alternatively arranged in a leading position.

In a further example, the plug connection additionally or alternatively comprises a releasable, mechanical lock which is configured to prevent removal of the frame insertion element from the frame after insertion of the frame insertion element into the frame, regardless of the orientation. In one variant, the detachable releasable lock for locking and releasing comprises a latching hook having a latching nose, a latching opening and a first actuating element, wherein in a rest position the latching hook engages in the latching opening in such a way that the latching hook projects through the latching opening and the latching nose engages at least partially behind an edge of the latching opening, in a pre-release position the latching hook engages at least partially in the latching opening in such a way that at least a part of the latching hook protrudes through the latching opening and the latching nose is released from the edge of the latching opening, in a release position the latching hook is arranged outside the latching opening, wherein the actuating element is configured such that actuation of the actuating element moves the latching hook and/or the latching opening from the pre-release position into the release position. In another variant, elements of the releasable mechanical lock are additionally or alternatively arranged symmetrically to the one or more mirror axes of the frame or the frame insertion element.

In another example, an electrical coupling pair additionally or alternatively comprises a socket as electrical contact and a pin insertable into the socket as electrical mating contact, wherein the pin is variable in length in the insertion direction and comprises a spring which is configured to counteract compression of the pin by means of a restoring force, and the socket and pin are arranged such that, in the connected state of the plug connection, the pin is compressed by the socket. This example of the coupling pair is typically advantageous in order to enable a secure electrical connection even in the event, for example, of vibrations and manufacturing tolerances of the coupling pairs.

In a variant of the control device comprising the releasable mechanical lock and the electrical coupling pair with spring, the spring used in the electrical coupling pair is configured so that the spring may form a restoring force that is greater than a force required to close the mechanical lock. This typically has the advantage that the frame insertion element is pushed out of the frame again by the spring if the lock does not engage. In another variant, a plurality of coupling pairs comprise a spring and the sum of the restoring forces of all springs is greater than the force required to close the mechanical lock.

In a further example of the control device, the plug connection additionally or alternatively comprises a sealing element which is arranged such that, in a connected state of the plug connection, the extent to which dust and/or water come into contact with the one or more electrical coupling pairs is reduced. In one variant, the seal prevents contact as far as possible.

In yet another example, an electrical coupling pair additionally or alternatively comprises a rod electrode as electrical contact and a rod socket as electrical mating contact, wherein the rod electrode has a plurality of rod contact elements along an axial direction of the rod electrode for independent transmission of two or more electrical variables, and the rod socket has a plurality of socket contact elements corresponding to the rod electrode along an axial direction of the rod socket. In one variant, the socket contact elements are configured as annular spring contacts and/or the rod contact elements are configured as cylindrical contact surfaces.

In a further example, an electrical coupling pair additionally or alternatively has an electrical collector and an electrical contact surface, wherein the electrical contact surface is arranged on a side of the frame insertion element pointing perpendicular to the insertion direction, and the electrical collector is arranged on an inner side of the frame corresponding to the electrical contact surface.

In another example, the frame and the frame insertion element additionally or alternatively have a basic shape in a plane perpendicular to the insertion direction, said basic shape being configured such that the frame insertion element is insertable into the frame in two orientations, which differ from each other by a relative rotation between frame and frame insertion element of 180° about an insertion direction.

In yet another example, contacts of the coupling pairs of the frame and the frame insertion element are additionally or alternatively arranged in a cluster-like manner within the frame or on the frame insertion element, wherein a distance between contacts of a cluster is smaller than a distance between the clusters.

In a further example, the plug connection additionally or alternatively comprises a guide element and a guided element, which are arranged on the frame and frame insertion element in such a way that, during insertion of the frame insertion element into the frame, the guide element guides the guided element along the insertion direction.

The blood pump device according to the present disclosure is now described below.

The blood pump device comprises an implantable blood pump and a control device according to one of the examples described above, wherein the control device is electrically connected to the blood pump. The blood pump device typically has the same advantages as the control device described above.

In one example of the blood pump device, the electrical connection between the blood pump and the control device is realized by cable. In another example, the electrical connection is additionally or alternatively realized wirelessly (for example, by induction). In further examples, the connection is additionally or alternatively used to control the blood pump and/or supply it with electrical energy.

In the following, examples of the control device and the blood pump device are explained with reference to the figures. First, an overview of the figures is given.

### BRIEF DESCRIPTION OF THE DRAWINGS

FIG. 1 shows an example of a control device for a blood pump which comprises an electronic control unit and an energy storage unit;

FIG. 2A shows a front view of the electronic control unit from FIG. 1, looking along an insertion direction;

FIG. 2B shows a front view of the energy storage unit from FIG. 1 with a view opposite the insertion direction;

FIG. 3A shows a front view of an arrangement of contacts of the control device from FIG. 1;

FIGS. 3B-E show alternative arrangements of contacts as compared to FIG. 3A;

FIG. 4A shows a cross section through the control device from FIG. 1 in a connected state along the insertion direction;

FIG. 4B shows a plug connection of the control device from FIG. 1 in the separated state;

FIG. 5 shows a control device with two rod electrodes and two rod sockets for establishing electrical contact;

FIG. 6 shows an enlargement of the rod electrodes and rod sockets of the control device from FIG. 5 in the connected state;

FIG. 7A shows a front view of an alternative electronic control unit with only one rod electrode;

FIG. 7B shows a front view of an energy storage unit corresponding to the electronic control unit shown in FIG. 7A with only one rod socket;

FIG. 8 shows an energy storage unit with a cluster-like arrangement of sockets; and

FIG. 9 shows a blood pump device.

### DETAILED DESCRIPTION

The following is a detailed description of the examples shown in the figures.

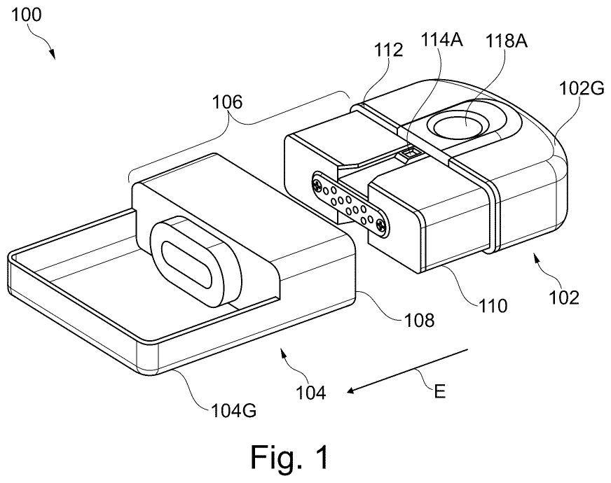

A first example of a control device is first described with reference to FIGS. 1-4.

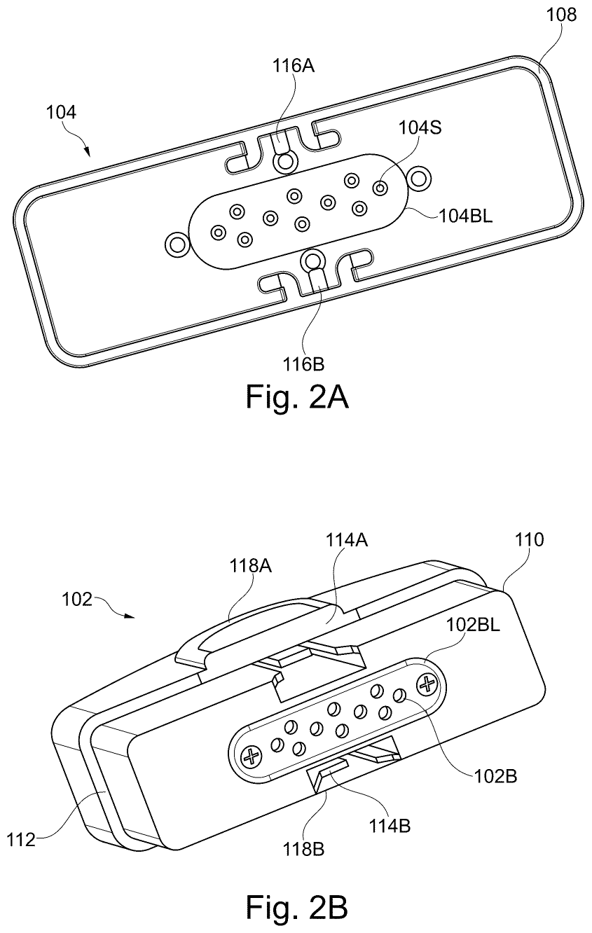

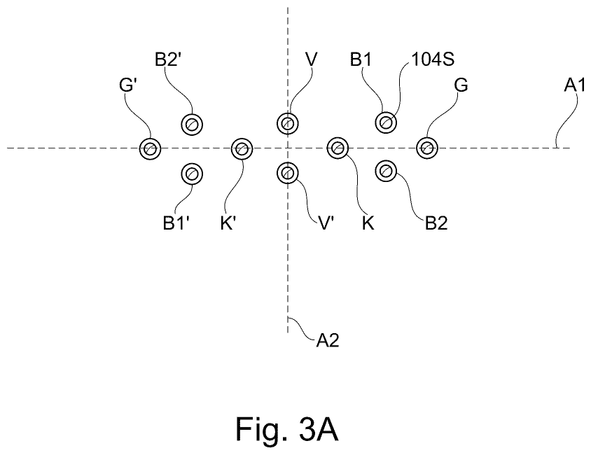

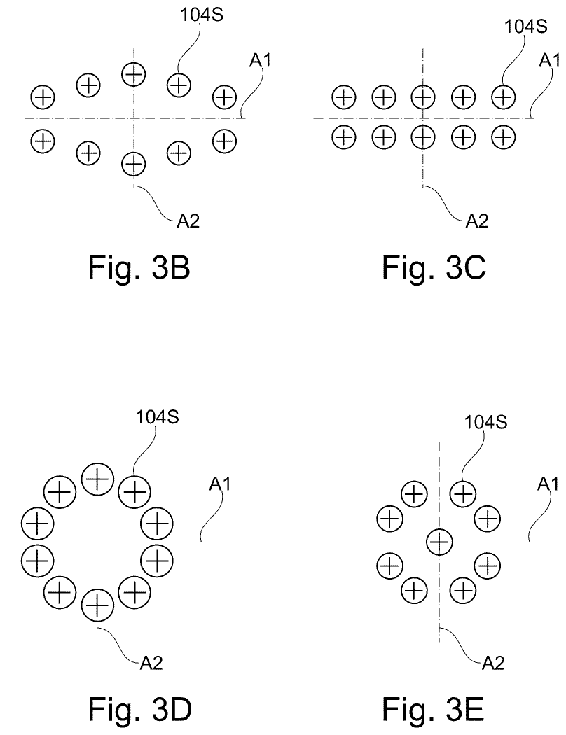

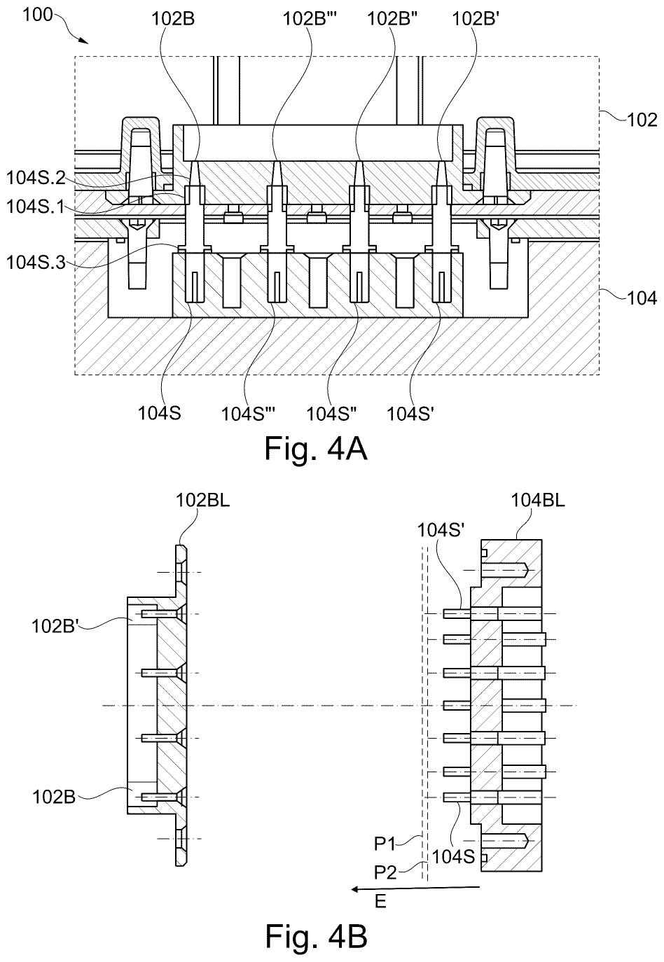

FIG. 1 shows an example of a control device 100 for a blood pump which comprises an electronic control unit 104 and an energy storage unit 102. FIG. 2A shows a front view of the electronic control unit 104 of FIG. 1 looking along the insertion direction E. FIG. 2B shows a front view of the energy storage unit 102 of FIG. 1 looking opposite the insertion direction E. FIG. 3A shows a front view of a plurality of contacts of the electronic control unit 104 of FIG. 1. FIG. 4A shows a cross section through the control device 100 of FIG. 1 in a connected state along the insertion direction E. Identical components of the control device 100 shown in FIGS. 1-4 are provided with identical reference signs in all figures.

The electronic control unit 104 and the energy storage unit 102 are detachably connectable to one another electrically and mechanically via a plug connection 106. The plug connection 106 comprises, for establishing a mechanical connection, a frame 108 and a frame insertion element 110 insertable into the frame along an insertion direction E, said frame and frame insertion element being formed by a housing 104G of the electronic control unit 104 and a housing 102G of the energy storage unit 102. Furthermore, the frame 108 and the frame insertion element 110 are configured such that the frame insertion element 110 is insertable into the frame 108 in two or more orientations which differ from one another by a relative rotation between the frame 108 and the frame insertion element 110 about the insertion direction E. In addition, the frame 108 and the frame insertion element 110 have a plurality of electrical coupling pairs for establishing an electrical connection between the electronic control unit 104 and the energy storage unit 102, and the plurality of electrical coupling pairs are arranged such that an electrical connection between the energy storage unit 102 and the electronic control unit 104 is possible in all orientations.

In the illustration in FIG. 1, only a part of the housing 104G is shown for simplification in order to make visible a rear side of a part of the electrical coupling pairs arranged on the electronic control unit 104. In the example shown, the housing 102G and the housing 104G are made of a thermoplastic material. Possible materials for the production of the plastic are ABS-PC, ABS, polyurethane and PMMA.

The frame 108 and the frame insertion element 110 have a rectangular basic shape and therefore have two orientations, which enable the frame insertion element 110 to be inserted into the frame 108—a first orientation and a second orientation, in which the electronic control unit 104 and the energy storage unit 102 are rotated 180° relative to each other about the insertion direction E.

The example of the control device 100 shown in FIGS. 1-4 comprises a total of 10 coupling pairs, wherein each coupling pair comprises a pin 104S arranged on the electronic control unit 104 and a socket 102B arranged on the energy storage unit 102. The arrangement of the pin 104S on the electronic control unit 104 and of the socket 102B on the energy storage unit 102 is not mandatory, i.e., the pin could also be arranged on the energy storage unit and the socket on the electronic control unit. However, the arrangement shown is advantageous for the safety of a user as well as for the protection of the device, since a contact on the energy storage unit 102 is located inside the socket 102B and is thus not directly accessible. The pins of the electronic control unit 104 are configured such that they may be inserted into the sockets of the energy storage unit 102 in order to establish an electrically conductive connection between the electronic control unit 104 and the energy storage unit 102. Furthermore, the pins are arranged on a rear wall within the frame 108 and the sockets are arranged on a front of the frame insertion element 110. The number of coupling pairs may be scaled as required and may therefore be adapted to the number of electrical contacts required for the respective application.

FIG. 3A again shows an enlarged view of the pins as they are arranged within the frame 108 of the electronic control unit 104. As is visible in FIG. 3A, the pins are arranged symmetrically with respect to mirror axis A1 and mirror axis A2. The mirror axes A1 and A2 correspond here to the mirror axes of the frame 108 in a plane perpendicular to the insertion direction E. Furthermore, the assignment of the coupling pairs is indicated in FIG. 3A by the reference signs B1, B2, G, K and V. The contacts B1 and B2 are used for bus communication, the contact G for a ground connection, the contact V for a voltage connection and the contact K for establishing a contact between electronic control unit 104 and energy storage unit 102. Each contact (and each mating contact on the energy storage unit 102) is formed here redundantly and is present twice, which is illustrated by the deleted reference signs B1′, B2′, G′ and K′. Furthermore, the redundant coupling pairs are arranged in such a way that an electrical connection corresponding to each contact is possible in all orientations from frame 108 to frame insertion element 110.

FIG. 3A shows only one possible arrangement of pins 104S. Other arrangements are also possible. FIG. 3B-E show alternative arrangements of the contacts. The arrangements shown in the figures are all symmetrical to the axes A1 and A2. In FIGS. 3B and 3C, the contacts are arranged in two rows above and below the axis A1. Whereas in FIG. 3C the pins 104S are arranged in a row parallel to the axis A1, in FIG. 3B the rows above and below the axis A1 are slightly curved. In FIG. 3D and FIG. 3E, the contacts are arranged in a circle around a point of intersection between the axis A1 and the axis A2. The arrangement shown in FIG. 3E also includes a contact that is arranged at the point of intersection between axis A1 and axis A2. This enables an uneven number of contacts with a symmetrical arrangement with respect to axis A1 and axis A2. Further arrangements are described later with reference to other examples.

The control device 100 furthermore comprises a detachable mechanical lock which is configured to prevent removal of the frame insertion element 110 from the frame 108 after insertion of the frame insertion element 110 into the frame 108, regardless of the orientation. In the example of FIGS. 1-4, the mechanical lock comprises two latching lugs 114A and 114B arranged on opposite sides of the frame insertion element 110, which, in a connected state, are configured to engage behind guide frame elements 116A and 116B, respectively, of a latching opening. Two actuating elements 118A and 118B arranged on opposite sides of the energy storage unit 102 are configured, when actuated, to release the mechanical connection between the latching nose and the respective frame element. In the region of the latching lugs 114A and 114B, there is also a notch that extends in the insertion direction. These notches may each serve as a guide element to guide frame elements 116A and 116B when the frame insertion element 110 is inserted into the frame 108.

In addition, the plug connection 106 comprises a sealing element 112 that extends around the frame insertion element 110 and cooperates with the frame 108 in a connected state of the plug connection 106 to reduce dust and water, for example, from coming into contact with the electrical coupling pairs.

FIG. 4A shows a cross section through the control device 100 along the insertion direction E in a connected state. It is shown how, in the connected state, the pins of the electronic control unit 104 are inserted into the sockets of the energy storage unit 102 in order to establish an electrically conductive connection. The cross section also shows further details of the pin structure. This structure is described for the pin 104S by way of example. The pin 104S is variable in length in the insertion direction and comprises a pin base 104S.3, which comprises a spring (not shown), which is configured to counteract a compression of the pin 104S by means of a restoring force. In the example shown, the length variability of the pin 104S is achieved by a telescopic rod-like structure of the pin 104S. For this purpose, the pin 104S comprises a sleeve 104S.1, which is axially aligned along the insertion direction E, and a tip 104S.2 arranged axially movably in the sleeve 104S.1. The tip 104S.2 is pressed by the spring in the pin base 104S.3 into an end of the sleeve 104S.1 facing away from the frame insertion element 110. Furthermore, the socket 102B and pin 104S are arranged such that the pin 104S is compressed by the socket 102B when the plug connection 106 is in the connected state, in order to allow an electrically conductive connection even with production tolerances. In addition, the springs used in the electrical coupling pairs are configured in such a way that the springs together form a restoring force that is greater than the force required to close the mechanical lock. This pushes the frame insertion element 110 out of the frame 108 if the mechanical lock is not yet properly engaged. This is typically advantageous so that a user may immediately recognize an error when connecting the frame 108 and frame insertion element 110.

FIG. 4B shows the plug connection from FIG. 4A in a separated state. In particular, FIG. 4B shows the plate element 102BL with the sockets and the plate element 104BL with the pins. In this figure, it is possible to see how pins 104S, 104S′ are arranged such that when the frame insertion element 110 is inserted into the frame 108, an electrical connection is established by one of the two coupling pairs before an electrical connection is established by the other pins, and when the frame insertion element 110 is removed from the frame 108, the electrical connection of the pins 104S and 104S′ is the last to be disconnected. This is achieved in the example 100 in which the pins 104S, 104S′ are arranged in a leading position in the insertion direction E with respect to the other pins, as is clear from the two planes P1 and P2 indicated in FIG. 4B. In other examples, not shown here, the sockets 102B and 102B′ are alternatively arranged in the leading position, whereby the same effect is achieved.

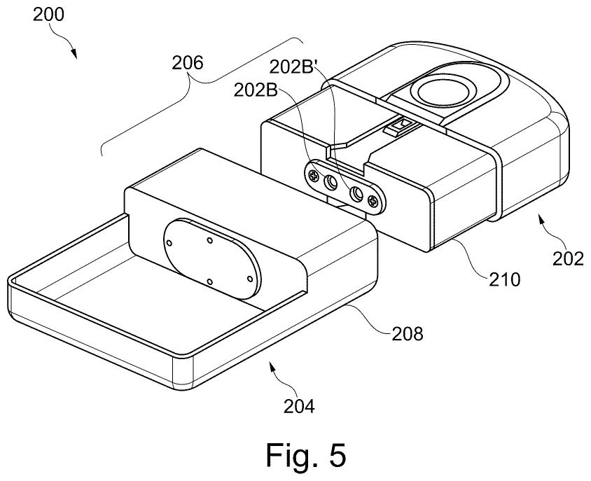

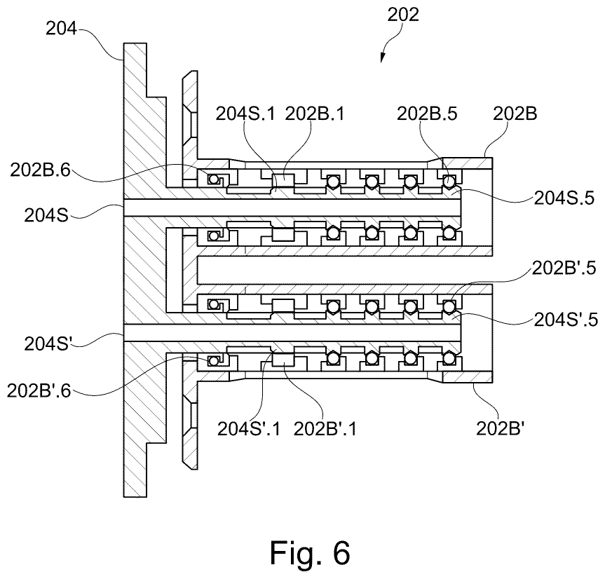

A further example of a control device is described below with reference to FIG. 5 and FIG. 6.

FIG. 5 shows a control device 200 with two rod electrodes 204S, 204S′ and two rod sockets 202B, 202B′ for establishing an electrical contact. FIG. 6 shows an enlargement of the rod electrodes 204S, 204S′ and rod sockets 202B, 202B′ of the control device 200 from FIG. 5 in the connected state.

The control device 200 is largely identical to the control device 100. The control device 200 comprises an energy storage unit 202 and an electronic control unit 204, which are mechanically and electrically detachably connected to each other via a plug connection 206. For this purpose, the plug connection 206 comprises a frame 208 arranged on the electronic control unit 204 and a frame insertion element 210 arranged on the energy storage unit 202. The control device 200 differs from the control device 100 primarily with regard to the establishment of an electrical connection between the electronic control unit 204 and the energy storage unit 202. For this purpose, the electronic control unit 204 comprises the two rod electrodes 204S and 204S′ arranged in the frame 208 and the two rod sockets 202B, 202B′ arranged on the frame insertion element 210. As shown in FIG. 6, the rod electrodes 202S, 202S′ comprise five cylindrical rod contact elements 204S.1-5 and 204S′.1-5, respectively, arranged axially one behind the other along a longitudinal axis of the rod electrodes. Each of the rod contact elements is arranged in such a way that they may establish an electrically conductive contact with one of five socket contact elements 202B.1-5, 202B′.1-5 arranged axially in the longitudinal direction. The socket contact elements 202B.1-5, 202B′.1-5 are formed here as annular spring contacts. Adjacent annular spring contacts are electrically insulated from each other by insulators between them. The annular spring contacts 202B.1-5, 202B′.1-5 are located in a housing made of a dielectric material such as plastic or ceramic. Furthermore, each of the rod sockets 202B, 202B′ comprises a seal 202B.6, 202B′.6 to prevent dust and liquids from entering the sockets in the connected state. The electrical contacts of the rod electrodes 204S, 204S′ and of the rod sockets 202B, 202B′ are each formed in such a way that a corresponding correct contact is established even when the electronic control unit 204 and the energy storage element are rotated by 180° about the insertion direction E relative to one another.

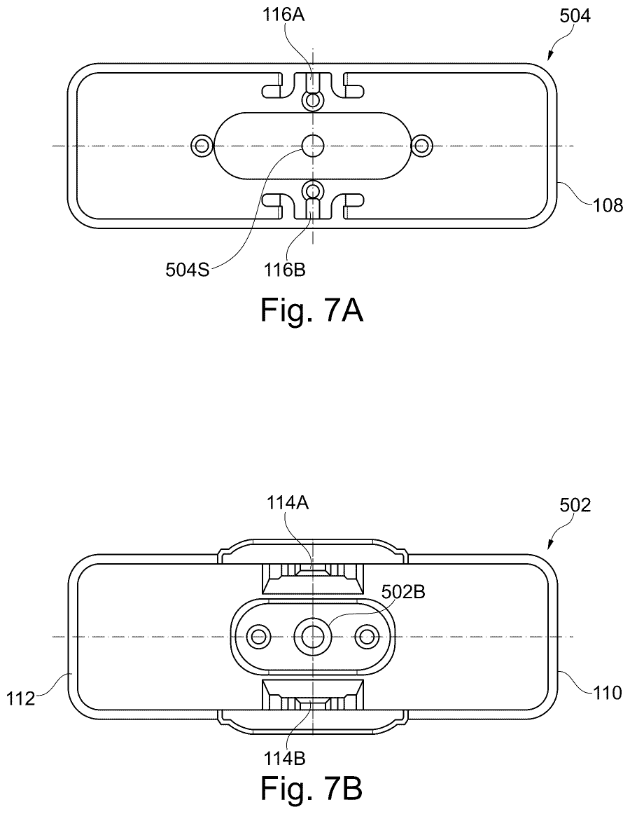

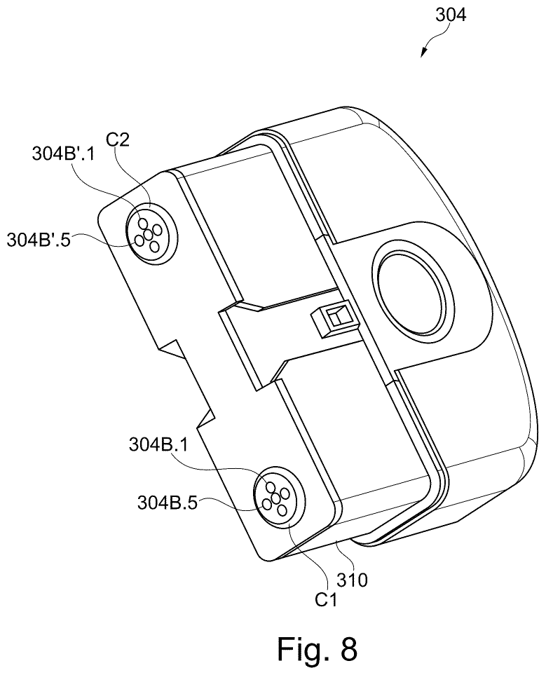

In the example 200 of the control device, two rod electrodes or rod sockets are used to create redundancy. Alternatively, it is also possible to use only one rod electrode. Such an example of a control device is shown in FIG. 7A and FIG. 7B.

FIG. 7A shows a front view of an alternative electronic control unit 504 with only one rod electrode 504S. FIG. 7B shows a front view of an energy storage unit 502 corresponding to the electronic control unit 504 shown in FIG. 7A with only one rod socket 502B. The rod electrode 504S and the rod socket 502B are constructed in the same way as the rod electrodes 204S, 204S′ and the rod sockets 202B, 202B′ discussed above. By arranging the rod electrode 504S or rod socket 502B centrally within the frame 108 or the frame insertion element 110, respectively, it is also possible here to bring the frame insertion element and the frame into electrical and mechanical contact with one another when the frame insertion element is rotated through 180° with respect to the frame 108 about the insertion direction E.

Other examples also have a different arrangement of contacts and mating contacts on the frame and frame insertion element. In the following, a further example will be described with reference to FIG. 8.

FIG. 8 shows an energy storage unit 304, with a cluster-like arrangement of sockets.

Analogously to other examples already discussed, the energy storage unit 304 comprises a frame insertion unit 310. Sockets 304B.1-5, 304B′.1-5 are arranged in two clusters C1 and C2 on the frame insertion unit 310. The clusters C1 and C2 are defined by the fact that the distance between contacts in a cluster is smaller than the distance between the clusters. In this example, the sockets themselves are also formed in such a way that an electrical connection is possible even when the energy storage unit and a corresponding electronic control unit are rotated relative to each other.

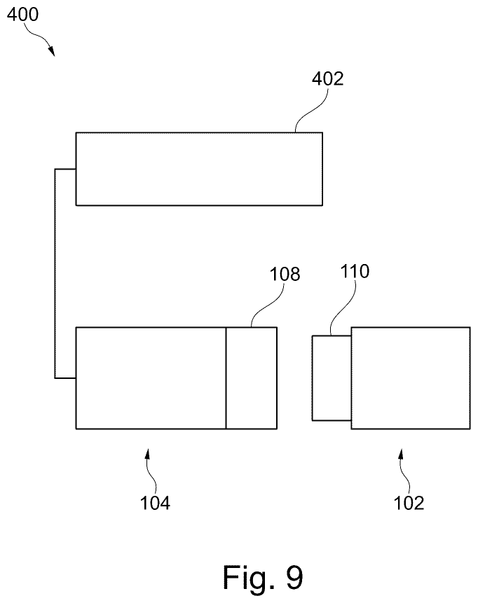

Lastly, the use of the control device presented as part of a blood pump device will be described with reference to FIG. 9.

FIG. 9 shows a blood pump device 400. The blood pump device 400 comprises the already described electronic control unit 104 with the frame 108 and the energy storage unit 102 with the frame insertion element 110. The electronic control unit 104 is electrically connected to a blood pump 402 in order to supply the blood pump 402 with electrical energy and to control it by means of control signals. However, as an alternative to the control device 100, any other of the control devices presented may also be part of the blood pump device.

The term module (and other similar terms such as unit, subunit, submodule, etc.) in the present disclosure may refer to a software module, a hardware module, or a combination thereof. Modules implemented by software are stored in memory or non-transitory computer-readable medium. The software modules, which include computer instructions or computer code, stored in the memory or medium can run on a processor or circuitry (e.g., ASIC, PLA, DSP, FPGA, or other integrated circuit) capable of executing computer instructions or computer code. A hardware module may be implemented using one or more processors or circuitry. A processor or circuitry can be used to implement one or more hardware modules. Each module can be part of an overall module that includes the functionalities of the module. Modules can be combined, integrated, separated, and/or duplicated to support various applications. Also, a function being performed at a particular module can be performed at one or more other modules and/or by one or more other devices instead of or in addition to the function performed at the particular module. Further, modules can be implemented across multiple devices and/or other components local or remote to one another. Additionally, modules can be moved from one device and added to another device, and/or can be included in both devices and stored in memory or non-transitory computer readable medium.

In summary, this disclosure describes a control device (100) for a blood pump, comprising an electronic control unit (104) and an energy storage unit (102) which are detachably connectable to one another electrically and mechanically via a plug connection (106). Here, the plug connection (106) for establishing a mechanical connection has a frame (108) and a frame insertion element (110) insertable into the frame (108) along an insertion direction (E), said frame and frame insertion element being formed by a housing (104G) of the electronic control unit (104) and a housing (102G) of the energy storage unit (102). Furthermore, the frame (108) and the frame insertion element (110) are configured such that the frame insertion element (110) is insertable into the frame (108) in two or more orientations which differ from one another by a relative rotation between the frame (108) and the frame insertion element (110) about the insertion direction (E). In addition, the frame (108) and the frame insertion element (110) have one or more electrical coupling pairs for establishing an electrical connection between the electronic control unit (104) and the energy storage unit (102), and the one or more electrical coupling pairs are arranged such that an electrical connection between the energy storage unit (102) and the electronic control unit (104) is possible in all orientations.

1. A blood pump control device, comprising:
an electronic control unit; and
an energy storage detachably connectable to the electronic control unit via a plug connection, wherein
the plug connection is configured to establish a mechanical connection and an electrical connection between the electronic control unit and the energy storage, and
the plug connection comprises:
a frame of a housing of the electronic control unit; and
a frame insertion element of a housing of the energy storage, which is insertable into the frame in an insertion direction in two or more orientations that differ by a relative rotation between the frame and the frame insertion element about the insertion direction, wherein
the frame and the frame insertion element have one or more electrical coupling pairs to establish the electrical connection, and
the one or more electrical coupling pairs are arranged to establish the electrical connection between the energy storage and the electronic control unit in the two or more orientations.

2. The blood pump control device according to claim 1, wherein
the frame and the frame insertion element each have one or more mirror axes in a plane perpendicular to the insertion direction, and
the electrical coupling pairs are arranged symmetrically with respect to the one or more mirror axes.

3. The blood pump control device according to claim 1, wherein
each coupling pair comprises an electrical contact arranged on the electronic control unit and a mating electrical contact arranged on the energy storage.

4. The blood pump control device according to claim 1, wherein
the plug connection is configured to transmit a plurality of different electrical quantities between the electronic control unit and the energy storage, and
the plug connection comprises at least two electrical coupling pairs for the transmission of each of the different electrical quantities.

5. The blood pump control device according to claim 1, wherein
the plug connection has at least two electrical coupling pairs configured such that a first electrical connection is established by one of the two coupling pairs when the frame insertion element is inserted into the frame before a second electrical connection is established by another coupling pair.

6. The blood pump control device according to claim 1, wherein the plug connection comprises:
a releasable mechanical lock configured to prevent removal of the frame insertion element from the frame after insertion of the frame insertion element into the frame regardless of orientation.

7. The blood pump control device according to claim 6, wherein the electrical coupling pair comprises:
a socket; and
a pin insertable into the socket, wherein
the pin is variable in length in the insertion direction,
the pin comprises a spring configured to counteract a compression of the pin via a restoring force, and
the socket and the pin are arranged such that the pin is compressed by the socket when the plug connection is in a connected state.

8. The blood pump control device according to claim 7, wherein
the spring used in the electrical coupling pair is configured such that the spring forms a restoring force that is greater than a force required to close the releasable mechanical lock.

9. The blood pump control device according to claim 1, wherein the plug connection further comprises:
a sealing element configured to reduce an extent to which dust and/or water come into contact with the one or more electrical coupling pairs in a connected state of the plug connection.

10. The blood pump control device according to claim 1, wherein the electrical coupling pair comprises:
a rod electrode; and
a rod socket, wherein
the rod electrode has a plurality of rod contact elements along an axial direction of the rod electrode for independently transmitting two or more electrical quantities, and
the rod socket has a plurality of socket contact elements along an axial direction of the rod socket corresponding to the rod electrode.

11. The blood pump control device according to claim 1, wherein the electrical coupling pair comprises:
an electrical collector; and
an electrical contact surface, wherein
the electrical contact surface is arranged on a side of the frame insertion element pointing perpendicularly to the insertion direction, and
the electrical collector is arranged on an inner side of the frame corresponding to the electrical contact surface.

12. The blood pump control device according to claim 1, wherein
the frame and the frame insertion element have a basic shape in a plane perpendicular to the insertion direction,
the basic shape is configured such that the frame insertion element is insertable into the frame in two orientations that differ from each other by a relative rotation of 180° between the frame and frame insertion element about an insertion direction.

13. The blood pump control device according to claim 1, wherein
contacts of the electrical coupling pairs of the frame and the frame insertion element are arranged in a cluster-like manner within the frame or on the frame insertion element, wherein
a distance between contacts of a cluster is smaller than a distance between the clusters.

14. A blood pump device comprising:
an implantable blood pump; and
the blood pump control device according to claim 1 electrically connected to the implantable blood pump.
